(19) United States
(12) Patent Application Publication
(10) Pub. No.: US 2011/0244539 A1
(43) Pub. Date: Oct. 6, 2011

(54) Selection Of Cellulolytic Microbes With High Growth Rates

(75) Inventors: Nicolai Panikov, Chestnut Hill, MA (US); Maria Sizova, Chestnut Hill, MA (US); Lee R. Lynd, Meriden, NH (US)

(73) Assignee: THE TRUSTEES OF DARTMOUTH COLLEGE

(21) Appl. No.: 13/121,383

(22) PCT Filed: Sep. 28, 2009
(86) PCT No.: PCT/US09/58597

§ 371 Date: Jun. 17, 2011

Related U.S. Application Data
(60) Provisional application No. 61/100,637, filed on Sep. 26, 2008.

(51) Int. Cl. C12P 7/10; C12N 1/20

(52) U.S. Cl. 435/165; 435/252.1; 435/252.7

(57) ABSTRACT

Methods for obtaining cellulolytic microbes with high growth rates are disclosed. For example, C. thermocellum colonies with growth rates higher than 0.17 hr″ 1 have been obtained by the present methods. In realizing higher growth rates, better bioprocessing efficiency can be achieved resulting in increased economy.

### CROSS-REFERENCE TO RELATED APPLICATIONS

This application claims priority to U.S. Patent Application No. 61/100,637, filed Sep. 26, 2008 and is incorporated herein by reference.

### GOVERNMENT INTERESTS

The United States government may have certain rights in the present invention as research relevant to its development was funded by The Department of Energy BioEnergy Science Center (BESC) contract number 60NANB1D0064.

### BACKGROUND

1. Field of the Invention

The present invention pertains to the field of biomass processing to produce ethanol and, more specifically, to the selection and use of thermophilic organisms with high growth rates.

2. Description of the Related Art

Cellulosic biomass represents an inexpensive and readily available raw material from which sugars may be produced. These sugars may be used alone or fermented to produce alcohols and other products. Among bioconversion products, interest in ethanol is high because it may be used as a renewable domestic fuel. Bioconversion processes are becoming economically competitive with petroleum fuel technologies. Various reactor designs, pretreatment protocols, and separation technologies are known, for example, as shown in U.S. Pat. Nos. 5,258,293 and 5,837,506.

One of the barriers impeding the establishment of a cellulosic biofuels industry is the recalcitrance of lignocellulose, that is, the difficulty of converting cellulosic biomass into soluble sugars. Various naturally cellulolytic microorganisms, such as Clostridium thermocellum (C. thermocellum) and Clostridium cellulolyticum (C. cellulolyticum), may be used to achieve this conversion. Strains of Thermoanaerobacterium thermosaccharolyticum and Thermoanaerobacterium saccharolyticum, which have substrate-utilizing capabilities that compliment those of C. thermocellum may also be used, includings strains that have been genetically modified to produce ethanol at high yields.

The rate of cellulose conversion is dependent on the growth rate of cellulolytic microorganisms. That is, the higher the growth rate of cellulolytic organisms, the faster the rate of cellulose conversion and, other things being equal, the smaller and less expensive the reaction vessel in which cellulose solubilization and fermentation of resulting sugars occurs. The growth rate for C. thermocellum on the cellulosic substrate Avicel, has been observed to be about 0.17 h⁻¹ (Lynd, L. R., P. J. Weimer, W. H. van Zyl, I. S. Pretorius. 2002. Microbial cellulose utilization: fundamentals and biotechnology. Microbiol. Mol. Biol. Rev. 66:506-577). It is desirable to obtain faster growing strains in order to decrease production costs.

### SUMMARY

The present instrumentalities advance the art and overcome the problems outlined above by providing methods for selection of microbes with growth rates higher than previously reported. The expectation of modest ability to increase the growth rate of cellulolytic microbes, for example, is based on the observation that cultures of C. thermocellum saturate (completely occupy) the substrate with cellulase. Efforts to increase the specific activity of cellulase enzymes systems via protein engineering have been unsuccessful. With the insoluble cellulose fully loaded with cellulase and with extensive efforts having failed to achieve meaningful increases in cellulase activity, the large increases disclosed herein are entirely unexpected. By realizing higher growth rates, it is anticipated that better bioprocessing efficiency can be achieved resulting in increased economy.

There are a number of industrial processes for the conversion of lignocellulosic materials for which the present invention would be suitable. These include, but are not limited to consolidated bioprocessing (CBP), simultaneous saccharification and fermentation (SSF) and simultaneous saccharification and co-fermentation (SSCF), involving both hexose and pentose-utilizing organisms.

In an embodiment, a method of selecting a bacterium comprises: culturing a bacterium on a solid medium until colonies are formed; selecting a bacterial colony that has one or more morphological characteristics associated with a subpopulation of C. thermocellum with a high growth rate; isolating the bacterial colony; and growing the bacterial colony in liquid broth to produce a bacterium with a high growth rate. In a more advanced embodiment, continuous culture in a pH auxostat—or similar continuous culture configuration that selects for microorganisms with high growth rates—is used to obtain cultures that exhibit a growth rate yet higher than both the original culture and strains obtained by isolating large colonies.

In one embobiment the bacterium used for selection can, for example be any one of the genus of Clostridium such as, but not limited to: Clostridium thermosulfurogenes, Clostridium cellulolyticum, Clostridium thermocellum, Clostridium thermohydrosulfuricum, Clostridium thermoaceticum, Clostridium thermosaccharolyticum, Clostridium tartarivorum, Clostridium thermocellulaseum, Clostridium thermolacticum, Clostridium hungatei; Clostridium phytofermentans; Clostridium cellulolyticum; Clostridium aldrichii; Clostridium termitididis.

### BRIEF DESCRIPTION OF THE DRAWINGS

FIG. 1A shows colonies of C. thermocellum on an agar-cellobiose MTC medium.

FIG. 1B shows a close-up view of the C. thermocellum of FIG. 1a.

FIG. 2 illustrates comparative growth rates of auxostat-selected, colony-selected and non-selected C. thermocellum.

FIG. 3 illustrates the development of faster-growing strains in a pH auxostat maintained at various pH values.

### DETAILED DESCRIPTION

There will now be shown and described a method for selecting cellulolytic microbes with high growth rates. As used herein, a “high growth rate” is a growth rate higher than 0.17 h⁻¹ for C. thermocellum and all other described cellulolytic microbes that grow optimally at 60 degrees C. or less or a growth rate higher than 0.4 h⁻¹ for Anaerocellum thermophilum.) In one aspect the growth rate for C. thermocellum is typically between 0.17 h⁻¹ and 0.50 h⁻¹ and more typically between 0.40 h⁻¹ and 0.50 h⁻¹.

Selection of large colonies, and in particular auxostat cultivation, was shown to be an effective means of obtaining cultures that exhibited growth rates on cellulose that were substantially higher than previously observed in C. thermocellum. These approaches may be generally utilized for cellulolytic microbes, such as but not limited to bacteria, genetically modified yeast, anaerobic and/or thermophilic bacteria, and Clostridium species.

It has been observed that there is considerable heterogeneity in cultures of C. thermocellum with respect to colony size, with some colonies exhibiting larger size than others. Colonies with sizes ranging from about 1 mm to about 3 mm were identified as those capable of producing the observed high growth rates. Cultures of C. thermocellum grown from the such colonies exhibited growth rates approximately substantially higher than four times faster than the control culture. A further substantial, nearly 2-fold, increase in growth rate was obtained by selection in auxostat culture. The ability to obtain growth rate increases of this magnitude was entirely unexpected and has no antecedent in the literature on cellulolytic microbes.

The observed growth rates of cellulolytic microbes, the presence of subpopulations with growth rates higher than previously described, and demonstration of methods to obtain microbes with high growth rates on cellulose are important in an applied context for the reasons outlined above.

As used herein the units of “growth rate” is the specific growth rate, μ, defined as (rate of cell formation)/(cell concentration). Typical units of the numerator in are g cells/L/hr. Typical units for the denominator are g cells/L. Hence, μ has units of 1/hr.

In an exponentially-growing batch culture, the cell concentration X is given by:

X=Xₒe^{μt},

where Xₒ is the initial cell concentration, and t is time.
By way of example, in order to determine the time necessary for doubling, td, we let X/Xₒ=2:

td=ln 2/μ.

### Example 1

### Selection of a High Growth-Rate Strain

### Materials and Methods

The strain Clostridium thermocellum (C. thermocellum) ATCC 27405 was used in the following examples. C. thermocellum is an anaerobic, thermophilic bacterium possessing cellulolytic and ethanogenic abilities that make it capable of directly converting a cellulosic substrate into ethanol.

C. thermocellum was maintained either in MTC medium with 3% Avicel, 3% cellobiose (Ozkan, Desai et al. 2001), or in a chemically-defined media (Johnson, Madia et al. 1981) modified as follows: cellobiose or cellulose, 10 g/L; KH₂PO₄, 4.25 g/L; (NH₄)₂SO₄, 2.1 g/L; MgCl₂.6H₂O, 1.0 g/L; CaCl₂.2H₂O, 0.15 g/L; FeSO₄.7H₂O, 0.002 g/L; Na-citrate, 3.0 g/L; L-cysteine, 1.0 g/L; rasazurin, 0.002 g/L; trace elements and vitamins. Medium was prepared in an anaerobic chamber with an atmosphere of CO₂/N₂/H₂ (10%/85%/5%).

Isolation of C. thermocellum was carried out by reactivating C. thermocellum from a frozen state in batch culture on MTC-cellobiose and then anaerobically plating on agar-cellobiose MTC. After about one week of incubation at 55° C. the colonies developed within the agar layer were examined and single spatially-separated colonies were transferred to fresh MTC medium. After isolation of individual colonies, the obtained clones were maintained without freezing at low positive temperatures (from 2 to 6° C.) on Avicel-containing MTC medium.

Batch and continuous fermentations on cellobiose (10 g/liter) and microcrystalline cellulose (Avicel PH 105, FMC, Philadelphia) (10 g/liter) were carried out in 2.5-liter round-bottom reactors (Sartorius A+) with agitation at 100 rpm and flow of ultra-pure N₂ (100 ml/min). The cultures were at constant pH 6.8 and 60° C. All cultivation parameters, including 1 N KOH titration rate, were logged to a computer. Every hour, 6.7 ml of culture were automatically pumped to a fraction collector (Waters) with simultaneous acidification to pH 1.5 with H₂SO₄ to stop metabolic activity.

The solids (residual cellulose and cells) were collected by centrifugation, washed, dried at 70° C., and then weighed. The outflow gases were analyzed by a CO₂ infrared gas analyzer (LiCor 800, Lincoln, Nebr.) and by mass-spectrometry using a RGA100 (Stanford Research System, CA). Three gas constituents —CO₂, H₂, and CH₄— were monitored and logged to a computer every 15 seconds. The liquid phase was analyzed by HPLC as described elsewhere (Zang and Lynd, 2005).

Continuous cultivation was performed in two regimes: chemostat (fixed dilution rate) and pH-auxostat. Having fixed pH of the fed medium (pH 8.5) the constant acidity of cultural liquid (pH 6.8) was electronically maintained by addition of fresh medium. Direct logging of the titration rate allowed for recordation of the instant specific growth as the algebraic sum of dilution rate and the apparent rate of biomass concentration.

Selection of High Growth-Rate C. thermocellum

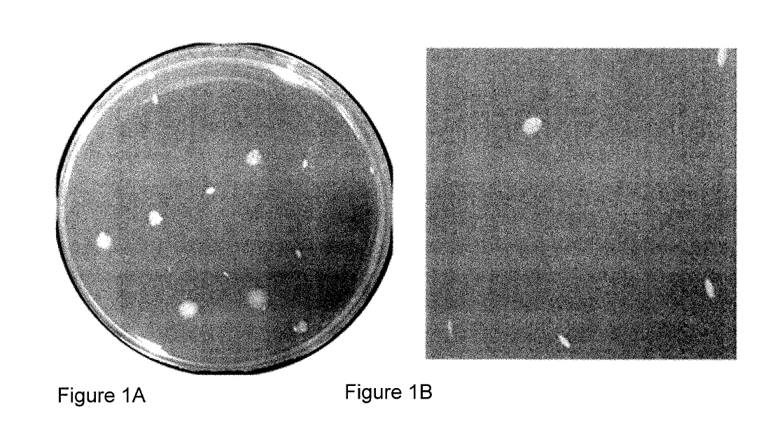

As described above, frozen C. thermocellum was reactivated, plated, incubated, separated, and transferred to fresh MTC medium. The colonies were heterogeneous in size, morphology and color. One set was large, round and bright-yellow. The sizes of the large colonies ranged from about 1 mm to about 5 mm. Another set was slim, oblong and pale-yellow (FIGS. 1A and 1B). The large bright-yellow colonies were cultured in MTC medium with 3% Avicel, 3% cellobiose, or the chemically-defined medium of Johnson (vide supra).

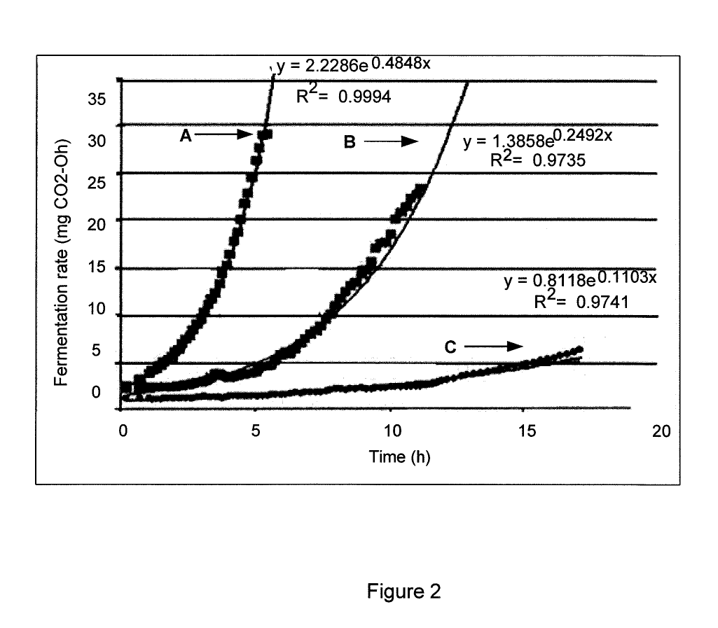

A C. thermocellum culture obtained from a large-colony isolate was maintained for two weeks in a pH-auxostat during which the growth rate was observed to continuously increase. Whereas the original culture exhibited a growth rate of 0.11 hr⁻¹, the large-colony isolate exhibited a growth rate of 0.28 hr⁻¹, and the auxostat-selected isolated exhibited a growth rate of 0.48 hr⁻¹. The growth rates were measured from the rate of CO₂ formation, which is proportional to biomass under the absence of a growth limitation. FIG. 2 shows the curves are a best-fit exponential regression, x=x₀exp(μt), where x₀ is initial cell mass, t is time, and μ is specific growth rate. The growth rates for the pH-auxostat pre-cultivated large colony-selected C. thermocellum (A), primary large colony-selected C. thermocellum (B) and non-selected C. thermocellum (C) were 0.48 h⁻¹, 0.28 h⁻¹, and 0.11 h⁻¹ respectively. In short, the pH-auxostat selected large colony-isolate of C. thermocellum had a growth rate ˜4.4 time greater than that of the non-selected C. thermocellum control in this experiment, the primary large colony C. thermocellum isolate had a growth rate ˜2.3 times greater than the non-selected C. thermocellum, and the auxostat-selected culture had a growth rate 1.7 times greater than the large colony isolate. The magnitude of these differences was entirely unexpected.

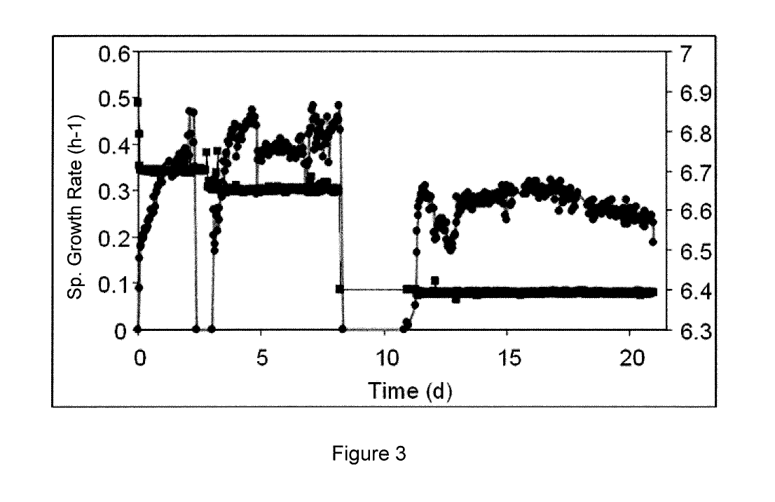

The effect of pH on growth rate was assessed on large colony C. thermocellum isolate maintained in an auxostat over a period of about 20 days. As shown in FIG. 3, cells initially grew at the rate of about 0.2 h⁻¹. However, as the culture evolved at constant pH between 6.7 and 6.8, the growth rate increased to 0.50 h⁻¹. At a lower pH of 6.4, the growth rate remained high between around 0.25 h⁻¹ and 0.30 h⁻¹. The culture was interrupted on day 3 and day 8 in order to test the ability of the fast strain to recover after perturbations.

The disclosed microbes may be utilized in a saccharification process, including a Simultaneous Saccharification and Fermentation (SSF) process as well as a consolidated bioprocessing (CBP) process with no added enzymes. Methods of utilizing cellulolytic microbes for the conversion of cellulosic material into ethanol are known. Cellulosic materials that may be converted by the presently described microbes include any feedstock that contains cellulose, such as wood, corn, corn stover, sawdust, bark, leaves, agricultural and forestry residues, grasses such as switchgrass or miscanthus or mixed prairie grasses, ruminant digestion products, municipal wastes, paper mill effluent, newspaper, cardboard or combinations thereof.

The description of the specific embodiments reveals general concepts that others can modify and/or adapt for various applications or uses that do not depart from the general concepts. Therefore, such adaptations and modifications should and are intended to be comprehended within the meaning and range of equivalents of the disclosed embodiments. It is to be understood that the phraseology or terminology employed herein is for the purpose of description and not limitation.

All references mentioned in this application are incorporated by reference to the same extent as though fully replicated herein.

1. A method of obtaining a fast-growing bacterium comprising:
culturing a bacterium on a solid medium until colonies are formed;
choosing a bacterial colony that has one or more morphological characteristics considered to contain a subpopulation with a high growth rate;
isolating the bacterial colony; and
growing the bacterial colony in liquid broth to produce a bacterium with a high growth rate.

2. The method of claim 1, wherein said bacterium comprises a thermophilic anaerobic bacterium.

3. The method of claim 1, wherein said bacterium comprises a cellulolytic bacterium.

4. The method of claim 1, wherein said bacterium comprises a species selected from the genus Clostridium.

5. The method of claim 1, wherein said bacterium comprises Clostridium thermocellum.

6. The method of claim 1 wherein the liquid broth has a pH ranging from 6.40-6.70.

7. The method of claim 1 wherein the liquid broth has a pH ranging from 6.65-6.70.

8. The method of claim 1 wherein the growth rate of said fast-growing bacterium is greater than 0.17 hr⁻¹.

9. The method of claim 1 wherein the growth rate of said fast-growing bacterium is at least 0.20 hr⁻¹.

10. The method of claim 1 wherein the growth rate of said fast-growing bacterium is at least 0.25 hr⁻¹.

11. The method of claim 1 wherein the growth rate of said fast-growing bacterium is at least 0.30 hr⁻¹.

12. The method of claim 1 wherein the growth rate of said fast-growing bacterium is at least 0.35 hr⁻¹.

13. The method of claim 1 wherein the growth rate of said fast-growing bacterium is at least 0.40 hr⁻¹.

14. The method of claim 1 wherein the growth rate of said fast-growing bacterium is at least 0.45 hr⁻¹.

15. The method of claim 1 wherein the growth rate of said fast-growing bacterium is at least 0.50 hr⁻¹.

16. The method of claim 1 wherein the growth rate of said fast-growing bacterium is between 0.17 hr⁻¹ and 0.50 hr⁻¹.

17. A bacterium selected for a high growth rate according to the method of claim 1.

18. The bacterium of claim 17 wherein the bacterium is a thermophilic anaerobic bacterium.

19. The bacterium of claim 17 wherein the bacterium is cellulolytic.

20. The bacterium of claim 17 wherein the bacterium is a species selected from the genus Clostridium.

21. The bacterium of claim 17 wherein the bacterium is Clostridium thermocellum.

22. The method of claim 1 wherein the step of culturing occurs in an auxostat.

23. A method for producing ethanol, said method comprising:
culturing a fast-growing bacterium obtained according to the method of claim 1 in a broth comprising a substrate of cellulosic material.

24. The method of claim 23, wherein said cellulosic material comprises a material selected from the group consisting of wood, corn, corn stover, sawdust, bark, leaves, agricultural and forestry residues, grasses, switchgrass, ruminant digestion products, municipal wastes, paper mill effluent, newspaper, cardboard and combinations thereof.

25. A method of obtaining a fast-growing bacterium, said method comprising:
culturing a bacterium on a solid medium until colonies are formed;
isolating a colony from said solid medium;
placing said colony in a liquid broth to form a culture;
placing the culture into a pH auxostat to select for said fast-growing bacterium.

26. The method of claim 25, wherein said bacterium comprises a thermophilic anaerobic bacterium.

27. The method of claim 25, wherein said bacterium comprises a cellulolytic bacterium.

28. The method of claim 25, wherein the bacterium comprises a species selected from the genus Clostridium.

29. The method of claim 25, wherein said bacterium comprises comprising Clostridium thermocellum.

30. The method of claim 25, wherein said liquid broth has a pH ranging from 6.40-6.70.

31. The method of claim 25, wherein said liquid broth has a pH ranging from 6.65-6.70.

32. The method of claim 25 wherein the growth rate of said fast-growing bacterium is greater than 0.17 hr⁻¹.

33. The method of claim 25 wherein the growth rate of said fast-growing bacterium is at least 0.20 hr⁻¹.

34. The method of claim 25 wherein the growth rate of said fast-growing bacterium is at least 0.25 hr⁻¹.

35. The method of claim 25 wherein the growth rate of said fast-growing bacterium is at least 0.30 hr⁻¹.

36. The method of claim 25 wherein the growth rate of said fast-growing bacterium is at least 0.35 hr⁻¹.

37. The method of claim 25 wherein the growth rate of said fast-growing bacterium is at least 0.40 hr⁻¹.

38. The method of claim 25 wherein the growth rate of said fast-growing bacterium is at least 0.45 hr⁻¹.

39. The method of claim 25 wherein the growth rate of said fast-growing bacterium is at least 0.50 hr⁻¹.

40. The method of claim 25 wherein the growth rate of said fast-growing bacterium is between 0.17 hr⁻¹ and 0.50 hr⁻¹.

41. The method of claim 25 wherein the growth rate of said fast-growing bacterium is between 0.40 hr⁻¹ and 0.50 hr⁻¹.

42. A method for producing ethanol, said method comprising:
culturing a bacterium on a solid medium until colonies are formed;
isolating a colony from said solid medium;
placing the colony in a liquid broth to form a culture;
placing the culture into a pH auxostat to select for a fast-growing bacterium;
culturing said fast-growing bacterium in a broth comprising a substrate of cellulosic material.

43. The method of claim 42, wherein said cellulosic material comprises a material selected from the group consisting of wood, corn, corn stover, sawdust, bark, leaves, agricultural and forestry residues, grasses, such as switchgrass, ruminant digestion products, municipal wastes, paper mill effluent, newspaper, cardboard and combinations thereof.
